(19) United States
(12) Patent Application Publication
(10) Pub. No.: US 2021/0251618 A1
(43) Pub. Date: Aug. 19, 2021

(54) IMPLANTABLE ATRIAL SEPTAL DEFECT OCCLUSION DEVICE WITH WOVEN CENTRAL SECTION ON LEFT ATRIAL FLANGE

(71) Applicant: Sree Chitra Tirunal Institute For Medical Sciences And Technology, Triruvananthapuram (IN)

(72) Inventors: Sujesh SREEDHARAN, Triruvananthapuram (IN); Jijo JERARD, Triruvananthapuram (IN); Liji Geetha VIJAYAN, Triruvananthapuram (IN); Bijulal SASIDHARAN, Triruvananthapuram (IN)

(73) Assignee: Sree Chitra Tirunal Institute For Medical Sciences And Technology, Triruvananthapuram (IN)

(21) Appl. No.: 17/253,443

(22) PCT Filed: Oct. 10, 2018
(86) PCT No.: PCT/IN2018/050642

§ 371 Date: Dec. 17, 2020

(30) Foreign Application Priority Data
Oct. 13, 2017 (IN) 201741036490

(51) Int. Cl. A61B 17/00; A61B 17/12

(52) U.S. Cl. CPC A61B 17/0057; A61B 2017/00867; A61B 2017/00615; A61B 17/12177

(57) ABSTRACT

The present invention relates to a device (D) for occluding atrial septal defect. Non-woven wires are introduced in one or more stages, with each stage interspaced with one or more braids and braided together with the wires from the woven centre and earlier stages to form the device (D). The transcatheter device (D) has two discs (101, 102) one a hub-less disc incorporating a woven central section on the left atrial side and the other disc on the right atrial side with a connecting neck (110) braided from wires. The device (D) has thrombogenic material in either discs (101, 102). A ridge (108) is configured on the periphery of either the left atrial disc or right atrial disc or both discs to improve structural stability of the device and provides enhanced elapsing force onto the septum reducing chances of migration.

### FIELD OF INVENTION

The present invention relates to implantable atrial septal defect occlusion device with woven central section on left atrial flange. More particularly, the invention relates to an implantable device employed for transcatheter closure of atrial septal defect.

### BACKGROUND OF THE INVENTION

Atrial septal defects are a type of congenital heart disease where the atrial wall or septum separating the right and left atria are not completely formed. To treat this condition, either it is surgically closed or can be closed percutaneously using closure devices. Closure devices comprise of a two part system where the device has a frame to support the device in place and a scaffold made of a thrombogenic substance to close the defect.

The first clinical devices had stainless steel frames supporting polyester patches to close the defect. Many patterns of closing the defects were available with different closure material and different retaining structures. Currently, shape memory alloy (SMA) and super-elastic materials are widely used in the frame. Use of Nitinol wire braided frame to form the device has wide acceptance because of its ease of delivery and better fixation.

Some of the prior art in this field detail about working on a plurality of metal strands heat treated to attain a shape and sustain it after it is collapsed into a catheter and later deployed (U.S. Pat. No. 5,846,261). U.S. Pat. No. 5,725,552 describes the method of forming a medical device from a plurality of metal wires by shaping and heat treatment using a mould. Such a device is said to be collapsible for passage through a catheter for deployment in a channel of a patient's body. U.S. Pat. No. 6,362,339B1 describes the method where a plurality of metal wires is heat treated to conform to the surface of a molding element to attain a predetermined shape which is a two lobed structure. Different techniques of forming the metal fabric are described in patent U.S. Pat. No. 9,179,899 B2 where it is formed by knitting wires and the structure is described as having proximal and distal portions which are orderly deployed in a particular unique way to close a cavity in the human body. Patents (US20070225760A1, DE102005053906A1) which claim a closed left atrial (LA) side use different braiding techniques to obtain a closed wall like formation on the LA side of the devices.

Braided designs of the wires are used to form a two lobed structure which while deploying, open on two sides of the defect closing it. The devices commonly have two lobes connected by a neck. The braids are held together by hubs on both proximal and distal ends with the distal end having suitable releasing mechanism to deploy the device. These hubs might cause a slight obstruction to normal blood flow. Also these hubs are the regions where it takes more time for endothelium formation.

Device migration and atrial roof erosion are two concerns with the present designs. There is a need for improvement of the device in terms of its structural stability and fixation and the present invention addresses the above need.

### Objects of the Invention

Therefore it is an object of the invention to propose an implantable atrial septal defect occlusion device made of braided metallic wires with a woven central section on its left atrial flange.

Another object of the invention is to propose an implantable atrial septal defect occlusion device with woven central section on left atrial flange which occupies less volume inside the left atrium by reducing the size of the hub or avoiding it altogether.

A still another object of the invention is to propose an implantable atrial septal defect occlusion device with woven central section on left atrial flange, which is capable of providing structural stability of the LA flange by using the novel woven central section.

A further object of the invention is to propose an implantable atrial septal defect occlusion device with woven central section on left atrial flange, which is able to provide better clasping strength on to the septal wall by introducing a ridge on the periphery of either the LA flange or the right atrial (RA) flange or both flanges thus increasing the migration resistance.

A still further object of the invention is to propose an implantable atrial septal defect occlusion device with woven central section on left atrial flange, which is capable of reducing atrial wall erosion, especially at the atrial roof, by employing a rounded ridge on the periphery of the flanges.

### SUMMARY OF INVENTION

A medical device which is the embodiment of the present invention for the closure of cardiac septal defects such as atrial septal defect is described here. The device is a braided collapsible and expandable structure with two flanges and a connecting neck in-between them, The neck fits into the septal defect with the flanges clamping on to the sides of the defect wall supporting it and at the same time maintaining a low profile to prevent any obstruction to normal blood flow. The low profile is maintained by use of a novel woven central section which also provides structural stability to the hub-less flange. The device comprises of metal mesh, such as a super-elastic metal mesh, shaped into the device with a thrombogenic material inside for closure of the defect. Further it has a ridge along the periphery of either the left atrial flange, or the right atrial flange or both flanges to enhance the fixation on the septal wall and provide a softer edge to reduce chances of atrial roof erosion.

This device scaffolds the remaining septal portion and helps in completing the atrial septum, thus preventing the intra-atrial blood flow. The device occupies minimal volume in the atria and provides minimum obstruction to blood flow in the heart chamber. The device is placed into the defect using a catheter and deployed on unsheathing from the catheter. The present invention improves the prior art in terms of minimising atrial volume occupied and providing better scaffolding by use of a woven central section on the left atrial disc and a ridge on its periphery; and reducing possible atrial wall erosion.

### BRIEF DESCRIPTION OF THE ACCOMPANYING DRAWINGS

FIG. 1 shows an embodiment of the invention showing RA side and a portion of LA side with the free ends of the wires inserted into a hub on the RA side.

FIG. 2(a) shows the representation of the twill weave which is done using the first fraction of the wires forming the device.

FIG. 2(b) shows arrangement of wires in the weave with corners opened.

FIG. 3 shows the pictorial representation of the LA side central section with the braids shown after the introduction of all the wires in three stages.

FIG. 4 shows the side view of the device showing the LA and RA sides and the neck region connecting the two discs.

FIG. 5 shows the isometric view of the invention showing ridge on the LA flange.

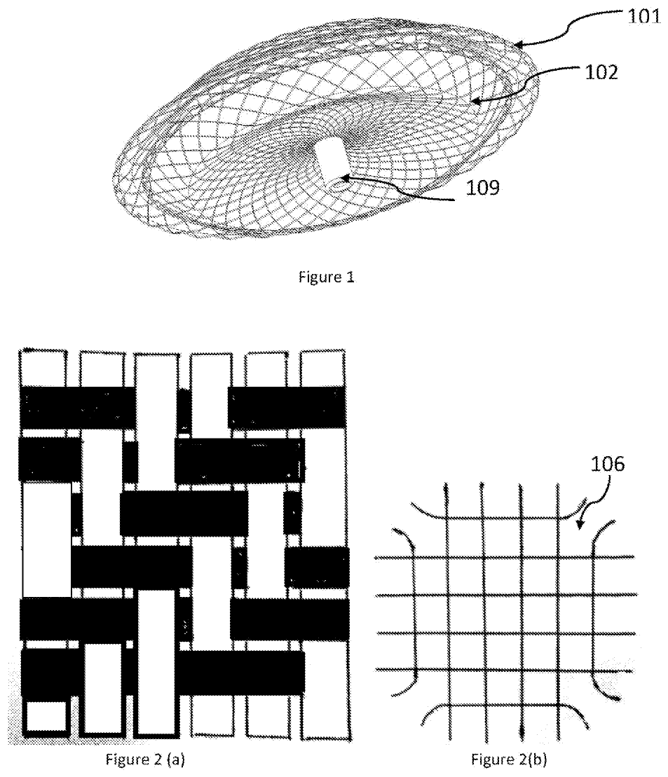

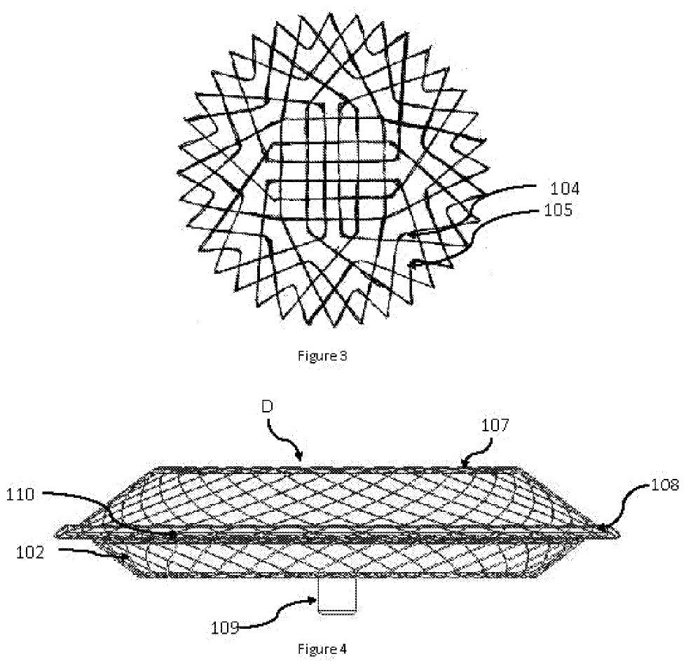

### DETAILED DESCRIPTION OF A PREFERRED EMBODIMENT OF THE INVENTION

The invention which is a device for occluding atrial septal defect is disclosed here.

A transcathether device for use as an atrial septal defect occluder has two discs, one a hub-less disc incorporating a woven central section on the left atrial side and the other disc on the right atrial side with a connecting neck braided from wires and the device has thrombogenic material in either discs and the said neck with a ridge along the periphery of either the left atrial disc or the right atrial disc or both discs.

Wires are braided and shaped into a two lobed structure and heat set into the required shape. Metal alloys such as Nitinol can be used to braid the device and shaped as is well known in the art. The braided structure has a closed end which forms the LA flange (101) of the device thus avoiding a hub and ensuring that minimum volume is occupied in the left atrium. The device having no hub and a flat central section on the left atrial disc occupying lesser volume inside the left atrium and results in improved endothelialization.

The closing of the LA flange has been achieved in the prior art either by (i) criss-crossing all the wires across the centre or (ii) criss-crossing only a part while looping back the remaining part in a particular pattern.

The method discussed here uses a novel combination of weaving the wires to form a mat like central section with one part of the wires, and then introducing the other parts in either a single stage or multiple stages, introducing them as loops. Looped wires are introduced in one or more stages, with each stage interspaced with one or more braids, and braided together with the wires from the woven centre and earlier stages to form the device. The next stages are introduced after one or more braids of the existing wires and finally all strands are braided together. The braid is a regular braid where as the weave could be regular or a twill weave, (105). The final braid could incorporate either a regular braid or a diamond braid.

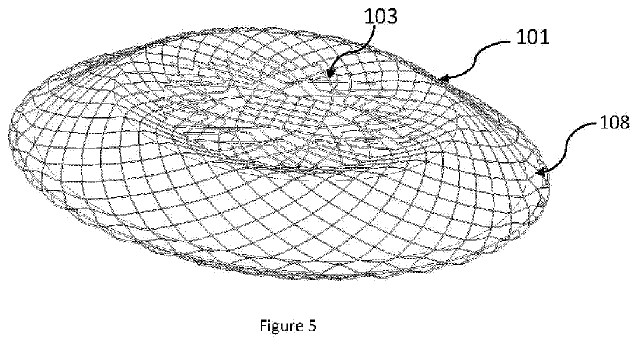

The woven wires are introduced onto a mandrel which holds the weave in place and the open ends are braided. Then the next stages of wires are added as loops, (104) after the formation of the braid between the woven wires. The newly introduced wires are braided with the existing braids forming a single braid; after two sets of braiding the final set of wires are added and braided on a suitable mandrel with closed end as visible in FIG. 5.

The second (104) and third (105) stages of wires are introduced onto the stubs on the mandrel which initially holds them in place before being braided onto the mandrel.

The wire mesh, (103) is formed with the weave mat (106) on the top and the wires introduced in subsequent stages (104 & 105), all getting braided together. The wire mesh is shaped into the required device shape of two flange structure with a connecting neck (110).

The weave design on the end results in the formation of a flat (107) end which helps in reducing the volume occupied by the device in the atrium. The hub-less flange can facilitate a continuous endothelium formation on top of the device. The metal mesh will be shaped into the twin-disc frame form and heat set; a material with thrombogenic properties, such as non-woven thin fabrics, is inserted into the twin discs and the neck region to induce closure of the atrial septal defect when the device is deployed in it.

The distal end where the wire ends (109) are combined together in a hub incorporating the release mechanism.

Further, a ridge (108) can be introduced on the periphery of either the LA flange or the RA flange, or both flanges to improve the structural stability of the device and provides enhanced clasping force onto the septum reducing chances of migration. The peripheral ridge gives a softer edge for the device reducing chances of atrial roof erosion.

We claim:

1. An implantable atrial septal defect occlusion device (D) with woven central section on left atrial flange, the said device (D) comprising;
two discs (101, 102), one a hub-less disc incorporating a woven central section on the left atrial side and the other disc configured on the right atrial side, with a connecting neck (110) braided from wires, the said discs consisting of thrombogenic material having a ridge (108), the said ridge disposed on the periphery of either the left atrial disc or the right atrial disc or both discs, wherein,
non-woven wires are configured in one or more stages, with each stage interspaced with one or more braids and braided together with the wires from the woven centre and earlier stages to form the device (D).

2. The device (D) as claimed in claim 1, wherein a ridge is configured on the periphery of either the LA flange or the RA flange or both flanges to improve the structural stability of the device and provides enhanced clasping force onto the septum reducing chances of migration wherein the said peripheral ridge gives a softer edge for the device reducing atrial wall erosion.

3. The device (D) as claimed in claim 1, wherein the said device having no hub and a flat central section on the left atrial disc occupying lesser volume inside the left atrium and improved endothelialization.

4. The device (D) as claimed in claim 1, wherein the braid is a regular braid and the central weave is a plain weave or a twill weave.

5. The device (D) as claimed in claim 1, wherein the distal end where the wire ends meet (109) are combined together in a hub incorporating the release mechanism.
